(19) United States
(12) Plant Patent Application Publication
(10) Pub. No.: US 2012/0227148 P1
(43) Pub. Date: Sep. 6, 2012

(54) BEGONIA PLANT NAMED 'BGDSU22-0'

(50) ;

(76) Inventor: Shinichi Nishimoto, Chikugo (JP)

(21) Appl. No.: 12/932,662

(22) Filed: Mar. 1, 2011

(51) Int. Cl. A01H 5/00

(52) U.S. Cl. PLT/345

(57) ABSTRACT

A new and distinct Begonia semperflorens cultivar named ‘BGDSU22-0’ is disclosed, characterized by unique, outwardly facing, large, semi double flowers, with white bracts and yellow tepals. Foliage is distinctively green to bronze, plants are flexible and resist breakage. The new variety is a Begonia, and is normally used as an ornamental garden or container plant.

### LATIN NAME OF THE GENUS AND SPECIES

Begonia semperflorens

### VARIETY DENOMINATION

‘BGDSU22-0’

### BACKGROUND OF THE INVENTION

The new cultivar is a product of a planned breeding program. The new variety originated as the result of a crossing performed in September 2004 of an unnamed, unpatented proprietary seed parent variety of Begonia semperflorens with the pollen parent, an unnamed, unpatented proprietary variety of Begonia semreflorens. The new variety was discovered by the inventor, Shinichi Nishimoto, a citizen of Japan, at a research greenhouse in Yama, Fukuoka, Japan, in September 2005.

Asexual reproduction of the new cultivar ‘BGDSU22-0’ was first performed in Yama, Fukuoka, Japan in March 2006. ‘BGDSU22-0’ has since produced several generations by vegetative cuttings and has shown that the unique features of this cultivar are stable and reproduced true to type. First sale and public access to the variety occurred May 2010. The variety was filed for Japanese Plant Breeder's Rights, Aug. 28, 2009 application Number 24036. There was no public access to the variety before May 2010.

### SUMMARY OF THE INVENTION

The cultivar ‘BGDSU22-0’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘BGDSU22-0.’ These characteristics in combination distinguish ‘BGDSU22-0’ as a new and distinct Begonia semperflorens cultivar:

1. Unique, large, semi double flowers with white bracts and yellow tepals

2. Bright, glossy dark green to bronze foliage

3. Flexible plant stems, resisting breakage.

4. Outwardly facing flowers.

### PARENT COMPARISON

Plants of the new cultivar ‘BGDSU22-0’ are similar to plants of the seed parent variety, in most horticultural characteristics. However, plants of the new cultivar ‘BGDSU22-0’ produce semi-double flowers, compared to fully double flowers produced by the seed parent. Additionally, the new variety produces dark green leaves, with a bronze edge, whereas the seed parent produces completely green foliage. The new variety is overall shorter than the seed parent.

Plants of the new cultivar ‘BGDSU22-0’ are similar to plants of the pollen parent variety, in most horticultural characteristics. However, plants of the new cultivar ‘BGDSU22-0’ produce semi-double flowers, compared to single flowers produced by the pollen parent. Additionally, the new variety produces larger flowers, larger leaves and more lateral branches than the pollen parent.

### COMMERCIAL COMPARISON

Plants of the new cultivar ‘BGDSU22-0’ are similar to plants of the patented commercial variety, Begonia semperflorens ‘Gum Drop White’ U.S. Plant Pat. No. 15,126 in most horticultural characteristics. However, plants of the new cultivar ‘BGDSU22-0’ produce flowers yellow tepals, and 6 to 8 bracts, whereas plants of ‘Gum Drop White’ produce flowers with white tepals, and 2 bracts. The new cultivar has a more bronze foliage coloration.

Plants of the new cultivar ‘BGDSU22-0’ are similar to plants of the patented commercial variety, Begonia semperflorens ‘Doublet White’ U.S. Plant Pat. No. 12,674 in most horticultural characteristics, including foliage color. However, plants of the new cultivar ‘BGDSU22-0’ produce flowers yellow tepals, and 6 to 8 bracts, whereas plants of ‘Doublet White’ produce flowers with white tepals, and 2 bracts. Additionally, the new variety produces larger flowers.

### BRIEF DESCRIPTION OF THE PHOTOGRAPH

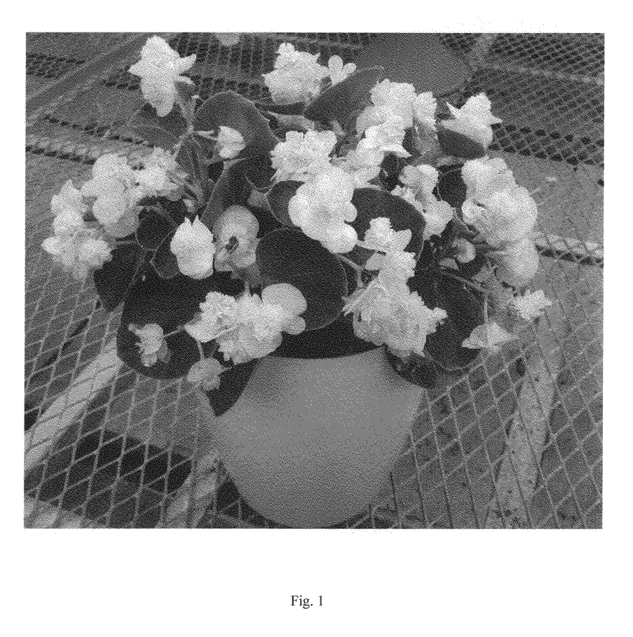

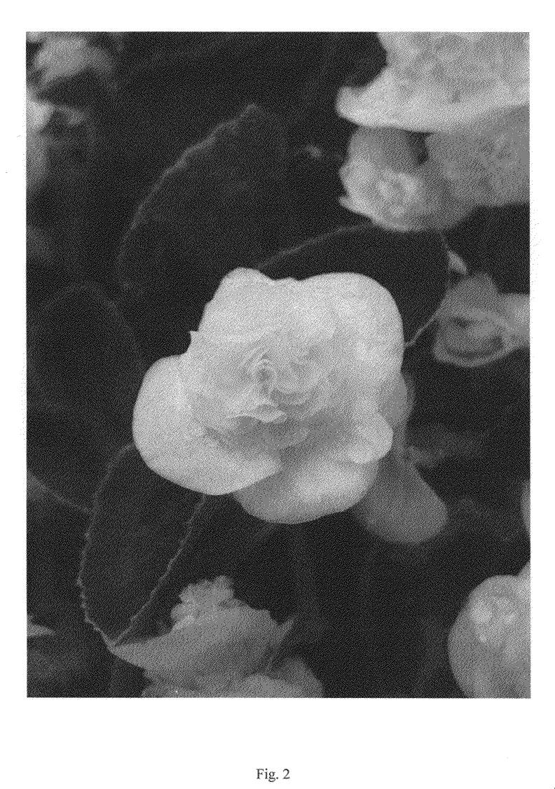

The accompanying photograph in FIG. 1 illustrates in full color a typical plant of ‘BGDSU22-0’ grown in a greenhouse. The pot size is a commercial 6 inch pot. The plant shown is approximately 4 months old. FIG. 2 illustrates a close-up view of the flowers and foliage. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2001, except where general terms of ordinary dictionary significance are used. The following observations and measurements describe ‘BGDSU22-0’ plants grown in a commercial controlled greenhouse in Oxnard, Calif. Temperatures ranged from 15° C. to 28° C. during the day and 18° C. to 22° C. at night No artificial light, photoperiodic treatments or chemical treatments were given to the plants. Measurements and numerical values represent averages of typical plant types.
Botanical classification: Begonia semperflorens ‘BGDSU22-0’
Age of plant described: Approximately 6 months.
Container size of plant described: 6 inch pot.
Propagation:
Type of propagation typically used: vegetative cuttings.
Time to roots: About 15 days at approximately 25° C.
Root description: Moderately dense, moderately branched, fine, fibrous, not fleshy, colored near RHS Grey-Green 196B
Plant:
Growth habit: Mounding, fleshy annual.
Pot size of plant described: 6 inch.
Height: Approximately 18 cm.
Plant spread: Approximately 28 cm.
Growth rate: Slow.
Branching characteristics: Moderate branching with lateral branches occurring from multiple basal branches.
Length of primary lateral branches: Approximately 10 cm.
Quantity of primary lateral branches: Approximately 20
Characteristics of primary lateral branches:
   Form.—Round in cross section.
   Diameter.—Approximately 0.5 cm.
   Color.—Near RHS Yellow-Green 146D.
   Texture.—Glabrous, shiny.
   Strength.—Moderately strong, fleshy, non-brittle.
Internode length: Approximately 1.2 cm on average.
Foliage:
   Orientation.—Upwardly facing foliage. Approximately 45° peduncle angle from stem.
   Strength.—Very strong, flexible, resists breakage.
   Texture.—Smooth, glabrous.
   Appearance.—Glossy.
   Arrangement.—Alternate.
   Quantity.—Approximately 6 per primary lateral branch.
   Average length.—Average 7.2 cm.
   Average width.—Average 5.1 cm.
   Shape of blade.—Broadly ovate to reniform.
   Apex.—Acute.
   Base.—Cordate.
   Margin.—Serrate.
   Texture of top surface.—Glabrous.
   Texture of lower surface.—Glabrous.
   Pubescence.—None.
   Aspect.—Upwardly cupped.
   Color.—Young foliage upper side: Near RHS Greyed-Orange 178B, with coloration influence from veins, near RHS Yellow-Green 147A. Margin near Yellow-Green N144A. Young foliage under side: Near RHS Greyed-Orange 178A, with coloration influence from veins, near RHS Yellow-Green 147A. Margin near Yellow-Green N144A. Mature foliage upper side: Near RHS Yellow-Green 147A with flush of Greyed-Orange 177A all over leaf. Margin near Yellow-Green N144A. Mature foliage under side: Near RHS Yellow-Green 148A with flush of Greyed-Red 178A all over leaf Margin near RHS Yellow-Green N144A.
   Venation.—Type: Palmate. Venation color upper side: Near RHS Yellow-Green 146C. Venation color under side: Near RHS Yellow-Green N144A.
   Petiole.—Length: Average Range Approximately 1 cm. Diameter: Approximately 0.3 cm. Color: Near RHS Yellow-Green 146C. Texture: Glabrous Stipule Description: Present and nearly all nodes, occurring 2 per node, non-fleshy, persistent. Length: Average 2.8 cm. Diameter: Average 1.3 cm. Color: Near RHS Yellow-Green 146C. Texture: Glabrous. Shape: Deltoid. Apex: Acute. Base: Cuneate.
Flower:
Flowering habit: Continuously flowering during Spring and Summer.
Days to flowering from rooted cutting: Approximately 2 weeks
Inflorescence and flower type and habit: Single flowers arranged in axillary cymes. Individual flowers composed of 6 to 8 large bracts and many center tepals. 2 bracts are significantly larger, symmetrical and opposite. Cymes held above foliage, approximately 2 to 3 open flowers per cyme. 2 cymes per terminal node.
Cyme size:
   Diameter.—Approximately 6.8 cm.
   Height.—Approximately 4.5 cm.
Flower longevity on plant: About 10 days.
Persistent or self-cleaning: Self-Cleaning.
Bud:
   Shape.—Flattened oval.
   Length.—Approximately 1.8 cm.
   Diameter.—Approximately 1.2 cm.
   Color.—Near RHS White 155C.
Flower size:
   Diameter.—Approximately 4.5 cm.
   Depth.—Approximately 3 cm.
Tepals:
   Quantity.—Approximately 120.
   Arrangement.—Rosette.
   Length.—A range of approximately 0.8 to 1.2 cm.
   Width.—Approximately 0.3 cm.
   Shape.—Spatulate.
   Apex.—Obtuse.
   Base.—Attenuate.
   Margin.—Entire.
   Aspect.—Undulating.
   Appearance, upper surfaces.—Matte.
   Appearance, lower surfaces.—Matte.
   Texture, upper surfaces.—Glabrous.
   Texture, lower surfaces.—Glabrous.
Color:
   When opening, upper surface.—Near RHS Yellow 4B.
   When opening, lower surface.—Near RHS Yellow 4B.
   Fully opened, upper surface.—Near RHS Yellow 4C.
   Fully opened, lower surface.—Near RHS Yellow 4C.
   Fading to, both sides.—Near RHS Yellow 4D.
Flower bracts:
   Quantity.—2 large bracts per flower, 4 to 6 smaller bracts.
Large bracts:
   Shape.—Broad orbicular.
   Length.—Approximately 2.1 cm.
   Diameter.—Approximately 2.8 cm.
   Apex.—Obtuse.
   Base.—Obtuse.
   Margin.—Entire.
   Texture.—Glabrous.
   Appearance.—Shiny.
   Aspect.—Flat with slight downward curve.
   Color, both sides.—Near RHS White N155A.
Small bracts:
   Shape.—Orbicular.
   Length.—Approximately 1.2 cm.
   Diameter.—Approximately 1.8 cm.
   Apex.—Obtuse.
   Base.—Obtuse.
   Margin.—Entire.
   Texture.—Glabrous.
   Appearance.—Shiny.
   Aspect.—Flat with slight downward curve.
   Color, both sides.—Near RHS White N155A.
Peduncles:
   Length.—Average Approximately 4.5 cm.
   Diameter.—Approximately 0.2 cm.
   Angle.—About 75° to the lateral branch.
   Strength.—Moderate, flexible.
   Texture.—Glabrous, shiny.
   Color.—Near RHS Yellow-Green 146D.
Pedicels:
   Length.—Average Approximately 1.9 cm.
   Diameter.—Approximately 0.1 cm.
   Angle.—About 45° to the peduncle.
   Strength.—Moderate, flexible.
   Texture.—Glabrous, shiny.
   Color.—Near RHS Green-White 157D.
Fragrance: Faint.
Reproductive organs: Flowers sterile, without male reproductive organs and highly reduced female organs
Pistil:
   Number.—1.
   Length.—Approximately 0.1 cm.
   Color.—Near RHS White N155A.
   Style.—Not present.
Other characteristics:
Seeds and fruits: Not produced.
Disease/pest resistance: Neither resistance nor susceptibility to diseases or pests has been observed in this variety.
Temperature tolerance: The new variety tolerates temperatures from approximately 5° to 35° C.

What is claimed is:

1. A new and distinct cultivar of Begonia plant named ‘BGDSU22-0’ as herein illustrated and described.
